(19) United States
(12) Patent Application Publication
(10) Pub. No.: US 2026/0260710 A1
(43) Pub. Date: Sep. 3, 2026

(54) RECORDING MEDIUM, INFORMATION PROCESSING METHOD, AND INFORMATION PROCESSING DEVICE

(71) Applicant: Fujitsu Limited, Kawasaki-shi (JP)

(72) Inventor: Naoki IIJIMA, Kawasaki (JP)

(73) Assignee: Fujitsu Limited, Kawasaki-shi (JP)

(21) Appl. No.: 19/547,330

(22) Filed: Feb. 23, 2026

(30) Foreign Application Priority Data
Feb. 28, 2025 (JP) 2025-031346

(51) Int. Cl. G16C 10/00

(52) U.S. Cl. CPC G16C 10/00

(57) ABSTRACT

A computer-readable recording medium stores therein a program for causing a computer to execute a process, the process includes calculating an energy of a molecule, based on an energy of each of a plurality of fragments obtained by dividing a structure of the molecule by a molecule dividing method, the calculating the energy including: calculating, for a first fragment among the plurality of fragments, a first problem using a first Hamiltonian corresponding to the first fragment, and calculating a first energy corresponding to the first fragment using a result of calculating the first problem; and calculating, for a second fragment among the plurality of fragments and corresponding to a second Hamiltonian identical or similar to the first Hamiltonian, a second energy corresponding to the second fragment using a result of calculating the first problem.

### CROSS REFERENCE TO RELATED APPLICATIONS

This application is based upon and claims the benefit of priority of the prior Japanese Patent Application No. 2025-031346, filed on Feb. 28, 2025, the entire contents of which are incorporated herein by reference.

### FIELD

The embodiments discussed herein are related to a recording medium, an information processing method, and an information processing device.

### BACKGROUND

Conventionally, in the field of drug discovery, material development, or the like, there is a quantum chemical calculation technique for analyzing the structure or properties of a molecule that is a candidate for a drug or a material. In the quantum chemical calculation, for example, the energy of a molecule is calculated. The energy is the ground or excitation energy. Here, in order to reduce the processing amount of the quantum chemical calculation, there is a molecule dividing method in which the structure of a molecule is split into multiple fragments, and the energy of each fragment is calculated and integrated to calculate the energy of the molecule. Examples of the molecular division method include Bootstrap Embedding (BE) and Density Matrix Embedding Theory (DMET).

In a related art, for example, a target system is divided into fragment units, and approximation using a monopole and a dipole is adopted for calculation of an electrostatic force according to a distance between fragments. In addition, for example, there is a technique of dividing a macromolecular system into fragments in which localized molecular orbitals may be constructed and which have a predetermined number of atoms or less. For example, refer to Japanese Laid-Open Patent Publication No. 2008-165508 and Japanese Laid-Open Patent Publication No. 2008-009706.

### SUMMARY

According to an aspect of an embodiment, a computer-readable recording medium stores therein a program for causing a computer to execute a process, the process includes calculating an energy of a molecule, based on an energy of each of a plurality of fragments obtained by dividing a structure of the molecule by a molecule dividing method, the calculating the energy including: calculating, for a first fragment among the plurality of fragments, a first problem using a first Hamiltonian corresponding to the first fragment, and calculating a first energy corresponding to the first fragment using a result of calculating the first problem; and calculating, for a second fragment among the plurality of fragments and corresponding to a second Hamiltonian identical or similar to the first Hamiltonian, a second energy corresponding to the second fragment using a result of calculating the first problem.

The object and advantages of the disclosure will be realized and attained by means of the elements and combinations particularly pointed out in the claims.

It is to be understood that both the foregoing general description and the following detailed description are exemplary and explanatory and are not restrictive of the disclosure.

### BRIEF DESCRIPTION OF DRAWINGS

FIG. 1 is an explanatory diagram depicting an example of an information processing method according to an embodiment.

FIG. 2 is an explanatory diagram depicting an example of an information processing system 200.

FIG. 3 is a block diagram of an example of a hardware configuration of an information processing device 100.

FIG. 4 is a block diagram depicting an example of a functional configuration of the information processing device 100.

FIG. 5 is an explanatory diagram depicting an operation example of the information processing device 100.

FIG. 6 is an explanatory diagram depicting an operation example of the information processing device 100.

FIG. 7 is an explanatory diagram depicting an example of similarity determination of embedded Hamiltonians.

FIG. 8 is an explanatory diagram depicting an example of an effect.

FIG. 9 is an explanatory diagram depicting an example of an effect.

FIG. 10 is a flowchart depicting an example of a procedure of an overall process.

FIG. 11 is a flowchart depicting an example of a procedure of a determination process.

FIG. 12 is a flowchart depicting an example of a procedure of a calculation process.

### DESCRIPTION OF EMBODIMENTS

First problems associated with the conventional techniques are discussed. However, in the related art, even when the molecule dividing method is used, it may be difficult to reduce the processing time necessary to perform the quantum chemical calculation. For example, as the scale of the structure of the molecule increases, the number of fragments obtained by dividing the structure of the molecule increases, and the processing time necessary for performing the quantum chemical calculation tends to increase.

Embodiments of a recording medium, an information processing method, and an information processing device according to the present disclosure will be explained below in detail with reference to the accompanying drawings.

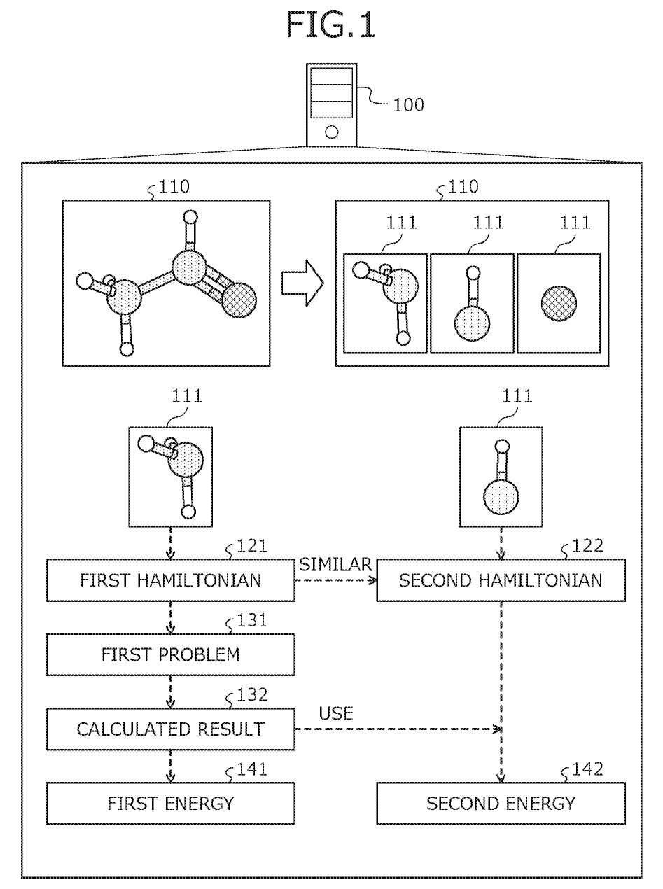

FIG. 1 is an explanatory diagram depicting an example of an information processing method according to an embodiment. The information processing device 100 is a computer for reducing the processing time necessary to perform quantum chemical calculation using a molecule dividing method. The information processing device 100 is, for example, a server or a personal computer (PC).

Conventionally, it is desired to perform quantum chemical calculation in the field of drug discovery, material development, or the like. The quantum chemical calculation is, for example, calculation of the energy of a molecule. The energy is the ground or excitation energy. Here, as the scale of the molecule increases, the processing time and the processing amount necessary for performing the quantum chemical calculation tend to increase. The scale is the number of atoms forming the molecule, etc.

Therefore, in order to reduce the amount of processing necessary when performing quantum chemical calculation, there is a molecule dividing method of dividing the structure of a molecule into multiple fragments, calculating the energy of each fragment, and integrating the calculated energies to calculate the energy of the molecule. The molecular splitting technique is, for example, Bootstrap Embedding (BE) or Density Matrix Embedding Theory (DMET).

However, even when the molecule dividing method is used, it may be difficult to reduce the processing time necessary for performing the quantum chemical calculation. For example, as the scale of the structure of the molecule increases, the number of fragments obtained by dividing the structure of the molecule increases, and the processing time necessary for performing the quantum chemical calculation tends to increase.

Therefore, in the present embodiment, an information processing method capable of reducing the processing time necessary for performing the quantum chemical calculation using the molecule dividing method will be described.

In FIG. 1, the information processing device 100 identifies each of multiple fragments 111 obtained by dividing a molecular structure 110. The information processing device 100 sets a Hamiltonian corresponding to each fragment 111. The Hamiltonian includes, for example, a term with a creation and/or annihilation operator (hereinafter, creation/annihilation operators).

The information processing device 100 calculates the energy of the molecule by calculating and integrating the energy of each fragment 111 by the molecule dividing method. For example, the information processing device 100 iteratively performs a process of calculating the energy of each fragment 111 until a predetermined exit condition is satisfied and calculates the energy of the molecule by integrating the energy of each fragment 111 calculated last. At this time, for example, the information processing device 100 calculates the energy of each fragment 111 as depicted in (1-1) and (1-2) below.

(1-1) The information processing device 100 calculates a first problem 131 using a first Hamiltonian 121 corresponding to a first fragment 111 for the first fragment 111 among multiple fragments 111. The first problem 131 is an eigenvalue problem. The first problem 131 is, for example, calculation process for obtaining an eigenstate corresponding to a wave function by solving the first Hamiltonian 121. The first problem 131 is, for example, an intermediate calculation process for calculating a first energy 141 corresponding to the first fragment 111. The information processing device 100 calculates the first energy 141 corresponding to the first fragment 111 using a result 132 of calculating the first problem 131. For example, the information processing device 100 calculates a reduced density matrix based on the eigenstates and calculates the first energy 141 corresponding to the first fragment 111 based on the calculated reduced density matrix. Accordingly, the information processing device 100 may appropriately calculate the energy corresponding to at least one fragment 111 of the multiple fragments 111.

(1-2) The information processing device 100 identifies among the multiple fragments 111, a second fragment 111 corresponding to a second Hamiltonian 122 that is the same as or similar to the first Hamiltonian 121. For example, the information processing device 100 identifies the second fragment 111 corresponding to the second Hamiltonian 122 in which all terms with creation/annihilation operators match those of the first Hamiltonian 121 and coefficients applied to the same terms as those of the first Hamiltonian 121 are the same as or similar to those of the first Hamiltonian 121. Accordingly, the information processing device 100 may identify the second fragment 111 considered to have a property similar to that of the first fragment 111.

The information processing device 100 calculates the second energy 142 corresponding to the second fragment 111 using the calculation result 132 of the first problem 131 without calculating the second problem using the second Hamiltonian 122 for the identified second fragment 111. For example, the information processing device 100 calculates the reduced density matrix based on the eigenstate that is the result 132 of calculating the first problem 131 and calculates the second energy 142 corresponding to the second fragment 111 based on the calculated reduced density matrix.

Accordingly, the information processing device 100 may reduce the processing time and the processing load necessary to calculate the second energy 142 corresponding to the second fragment 111. As described above, the information processing device 100 may reduce the processing time necessary to perform the quantum chemical calculation using the molecule dividing method.

Here, while a case where functions as the information processing device 100 are realized by a single computer has been described, the present disclosure is not limited hereto. For example, functions of the information processing device 100 may be realized by cooperation of multiple computers. For example, functions of the information processing device 100 may be implemented on a cloud.

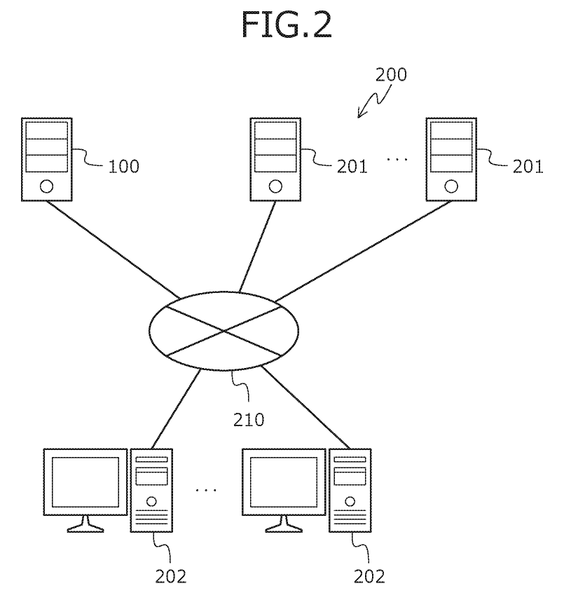

Next, an example of an information processing system 200 to which the information processing device 100 depicted in FIG. 1 is applied will be described with reference to FIG. 2.

FIG. 2 is an explanatory diagram depicting an example of the information processing system 200. In FIG. 2, the information processing system 200 includes the information processing device 100, one or more chemical calculating devices 201, and one or more client devices 202.

In the information processing system 200, the information processing device 100 and the chemical calculating devices 201 are connected via a wired or wireless network 210. The network 210 is, for example, a local area network (LAN), a wide area network (WAN), the Internet, or the like. In the information processing system 200, the information processing device 100 and the client devices 202 are connected via the wired or wireless network 210.

The information processing device 100 is a computer for performing quantum chemical calculations. The information processing device 100 obtains a processing request requesting execution of quantum chemical calculation for a molecule of interest by using the molecule dividing method. The molecule dividing method is, for example, DMET. Quantum chemical calculations include, for example, calculating the energy of a molecule of interest. The energy is, for example, ground energy or excitation energy.

The processing request includes, for example, structural information indicating the structure of the molecule of interest. The structural information includes, for example, coordinates of each atom among multiple atoms forming the molecule of interest. The structural information includes, for example, the type of each of the atoms forming the molecule of interest. The processing request may include, for example, a division number indicating how many fragments the structure of the molecule of interest is to be divided into. The number of divisions may be set in advance by the user, for example.

The information processing device 100 identifies the structure of the molecule of interest based on the structure information included in the processing request. The information processing device 100 generates fragment information indicating each fragment by dividing the structure of the identified molecule of interest into multiple fragments corresponding to the number of divisions. Based on the fragment information, the information processing device 100 expands DMET and performs quantum chemical calculation for calculating the energy of the molecule of interest.

For example, the information processing device 100 iteratively performs a series of processes of calculating the energy of each fragment until a predetermined exit condition is satisfied. The predetermined exit condition is, for example, that the sum of the number of atoms of each fragment matches the number of atoms of the molecule.

The series of processes includes, for example, a first process of calculating a first problem using a first Hamiltonian corresponding to the first fragment and calculating a first energy corresponding to the first fragment using a result of calculating the first problem. The first problem is, for example, an eigenvalue problem. The first problem is, for example, a calculation process of obtaining an eigenstate corresponding to a wave function by solving the first Hamiltonian. The series of processes includes, for example, a second process of using a result of calculating the first problem and thereby calculating a second energy corresponding to a second fragment corresponding to a second Hamiltonian that is the same as or similar to the first Hamiltonian.

The series of processes includes, for example, a third process of updating the Hamiltonian of each fragment when a predetermined exit condition is not satisfied when the energy of each fragment is calculated. For example, the information processing device 100 may perform a series of processes in cooperation with the chemical calculating devices 201. Specifically, the information processing device 100 performs the first process in cooperation with the chemical calculating devices 201 by controlling the chemical calculating devices 201 so as to solve the first problem.

When a predetermined exit condition is satisfied, the information processing device 100 calculates the energy of the molecule of interest, based on the energy of each fragment calculated last. The information processing device 100 outputs the calculated energy of the molecule of interest as a result of performing the quantum chemical calculation on the molecule of interest. The output format is, for example, display on a display, print output to a printer, transmission to another computer, or storage in a storage area. The other computer is, for example, one of the client devices 202. For example, the information processing device 100 may output the calculated energy of the molecule of interest as a result of performing the quantum chemical calculation on the molecule of interest so that the user may refer to the energy. The information processing device 100 is, for example, a server or a PC.

Each chemical calculating device 201 is a computer that performs quantum chemical calculation with respect to molecules. The chemical calculating device 201 calculates the first problem under the control of the information processing device 100. For example, the chemical calculating device 201 may calculate the first problem in cooperation with another chemical calculating device 201. The chemical calculating device 201 transmits a result of calculating the first problem to the information processing device 100. The chemical calculation device 201 is, for example, a server or a PC. The chemical calculation device 201 may include a quantum simulator. The chemical calculation device 201 may be, for example, an actual machine of a quantum computer.

The client device 202 is a computer utilized by a user who desires to perform a quantum chemical calculation on a molecule of interest. The user is, for example, an operator. The client device 202 generates a processing request that requests execution of the quantum chemical calculation for the molecule of interest using the molecule dividing method in response to the operation input of the user. The client device 202 obtains structure information indicating the structure of the molecule of interest, for example, in response to an operation input by the user. The client device 202 generates, for example, a processing request including structural information indicating the structure of the molecule of interest.

The client device 202 transmits the generated processing request to the information processing device 100. The client device 202 receives, from the information processing device 100, a result of performing the quantum chemical calculation on the molecule of interest. The client device 202 outputs the result of performing the quantum chemical calculation on the molecule of interest so that the user may refer to the result. The client device 202 is, for example, a PC, a tablet terminal, or a smartphone.

Here, while a case in which the information processing device 100 is a device different from the chemical calculating device 201 has been described, the present disclosure is not limited hereto. For example, the information processing device 100 may have a function of the chemical calculating device 201 and may also operate as the chemical calculating device 201. In this case, the information processing system 200 may omit the chemical calculating device 201.

Here, while a case in which the information processing device 100 is a device different from the client device 202 has been described, the present disclosure is not limited hereto. For example, the information processing device 100 may have a function of the client device 202 and may also operate as the client device 202. In this case, the information processing system 200 may omit the client device 202.

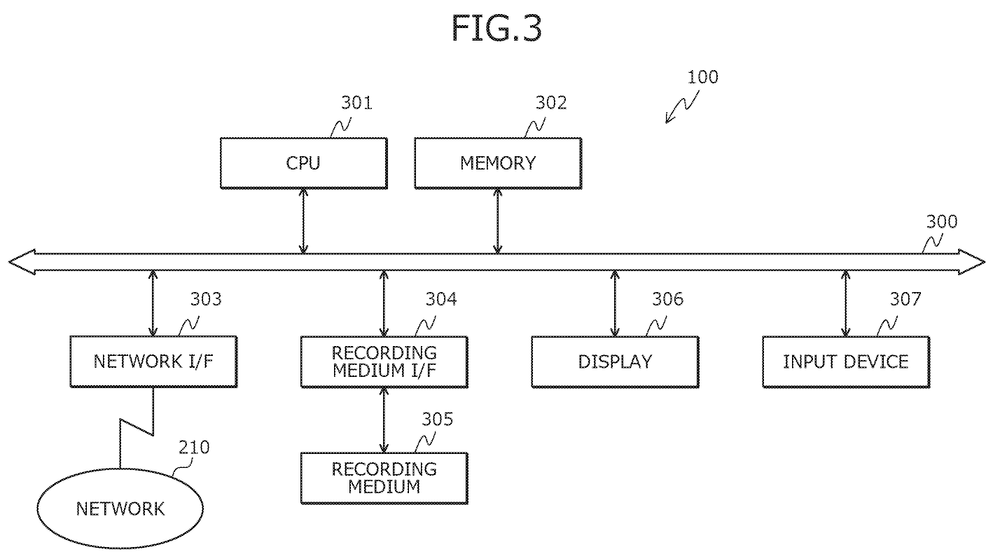

Next, with reference to FIG. 3, an example of a hardware configuration of the information processing device 100 is described.

FIG. 3 is a block diagram of an example of the hardware configuration of the information processing device 100. In FIG. 3, the information processing device 100 has a central processing unit (CPU) 301, a memory 302, and a network interface (I/F) 303. The information processing device 100 also has a recording medium I/F 304, a recording medium 305, a display 306, and an input device 307. Further, the components are connected to each other by a bus 300.

Here, the CPU 301 governs overall control of the information processing device 100. The memory 302, for example, includes a read-only memory (ROM), a random-access memory (RAM), and a flash-ROM. In particular, for example, the flash-ROM and/or ROM stores therein various programs and the RAM is used as a work area of the CPU 301. Programs stored to the memory 302 are loaded onto the CPU 301, whereby encoded processes are executed by the CPU 301.

The network I/F 303 is connected to the network 210 via a communications line and is connected to other computers through the network 210. Further, the network I/F 303 administers an internal interface with the network 210 and controls the input and output of data with respect to the other computers. The network I/F 303, for example, is a modem, a LAN adapter, or the like.

The recording medium I/F 304 controls the reading and writing of data with respect to the recording medium 305 under the control of the CPU 301. The recording medium I/F 304 is, for example, a disk drive, a solid-state drive (SSD), a universal serial bus (USB) port, or the like. The recording medium 305 is a nonvolatile memory storing data written thereto under the control of the recording medium I/F 304. The recording medium 305 is, for example, a disk, a semiconductor memory, a USB memory, or the like. The recording medium 305 may be removable from the information processing device 100.

The display 306 displays data such as a cursor, icons, toolboxes, documents, images, or functional information. The display 306 is, for example, a cathode ray tube (CRT), a liquid crystal display, or an organic electroluminescence (EL) display. The input device 307 includes keys for inputting characters, numbers, or various instructions, and inputs data. The input device 307 is, for example, a keyboard or a mouse. The input device 307 may be, for example, a touch panel-type input pad, a numeric keypad, or the like.

The information processing device 100 may include, for example, a camera in addition to the above-described components. Further, the information processing device 100 may include, for example, a printer, a scanner, a microphone, a speaker, or the like in addition to the above-described components. The information processing device 100 may include, for example, the recording medium I/F 304 and/or the recording medium 305 in plural. The information processing device 100 may omit, for example, the display 306 and/or the input device 307. The information processing device 100 may omit the recording medium I/F 304 and the recording medium 305, for example.

An example of a hardware configuration of the chemical calculating devices 201 is, for example, similar to the example of the hardware configuration of the information processing device 100 depicted in FIG. 3 and thus, description thereof is omitted.

An example of a hardware configuration of the client devices 202 is, for example, similar to the hardware configuration example of the information processing device 100 depicted in FIG. 3, and thus, description thereof is omitted.

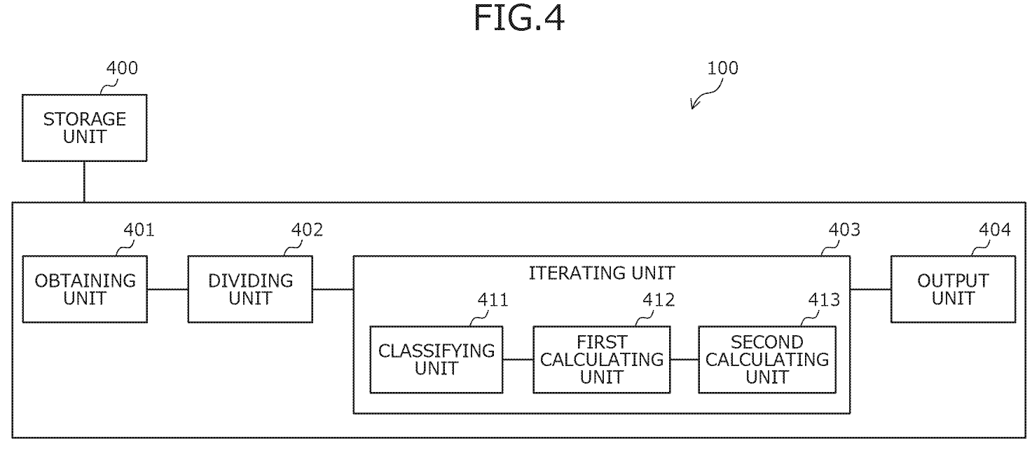

Next, an example of a functional configuration of the information processing device 100 will be described with reference to FIG. 4.

FIG. 4 is a block diagram depicting an example of a functional configuration of the information processing device 100. The information processing device 100 includes a storage unit 400, an obtaining unit 401, a dividing unit 402, an iterating unit 403, and an output unit 404. The iterating unit 403 includes a classifying unit 411, a first calculating unit 412, and a second calculating unit 413.

The storage unit 400 is realized by, for example, a storage area such as the memory 302 or the recording medium 305 depicted in FIG. 3. Hereinafter, while a case where the storage unit 400 is included in the information processing device 100 will be described, the present disclosure is not limited hereto. For example, the storage unit 400 may be included in a device different from the information processing device 100, and the storage content of the storage unit 400 may be referable from the information processing device 100.

The obtaining unit 401 to the output unit 404 function as an example of a controller. Specifically, the functions of the obtaining unit 401 to the output unit 404 are realized, for example, by causing the CPU 301 to execute a program stored in a storage area such as the memory 302 or the recording medium 305 depicted in FIG. 3 or by the network I/F 303. The processing result of the functional units is stored to, for example, a storage area such as the memory 302 or the recording medium 305 depicted in FIG. 3.

The storage unit 400 stores various types of information referred to or updated in the processes of each functional unit. The storage unit 400 stores, for example, structural information indicating the structure of a molecule of interest. The structural information includes, for example, coordinates of each atom among multiple atoms forming the molecule of interest. The structural information includes, for example, a type of each of the atoms forming the molecule of interest. The structural information includes, for example, an atomic index of each of the atoms forming the molecule of interest. The structure information is obtained by, for example, the obtaining unit 401. The structure information may be set in advance by the user, for example.

The storage unit 400 stores, for example, a basis function system. The basis set is a set of functions representing molecular orbitals. The basis function system is, for example, cc-pV5Z, cc-pVQZ, cc-pVTZ, cc-pVDZ, or STO-3G. The basis function system is obtained by, for example, the obtaining unit 401. The basis function system may be set in advance by a user, for example.

The storage unit 400 stores, for example, the number of divisions. The number of divisions indicates, for example, how many fragments the structure of the molecule of interest is divided into. The number of divisions is, for example, the number of fragments. The division number is obtained by the obtaining unit 401, for example. The number of divisions may be set in advance by the user, for example.

The storage unit 400 stores, for example, fragment information indicating each of multiple fragments obtained by dividing the structure of the molecule of interest. The fragment information includes, for example, an index of each atom of one or more atoms belonging to the fragment among the multiple atoms forming the molecule of interest. The fragment information is generated by, for example, the dividing unit 402. The fragment information may be obtained by the obtaining unit 401, for example. The fragment information may be set in advance by the user, for example.

The obtaining unit 401 obtains various types of information used for the processes of the functional units. The obtaining unit 401 stores the obtained various types of information to the storage unit 400 or outputs the obtained various types of information to the functional units. In addition, the obtaining unit 401 may output various types of information stored in the storage unit 400 to the functional units. The obtaining unit 401 obtains various types of information based on, for example, an operation input of a user. For example, the obtaining unit 401 may receive various types of information from a device different from the information processing device 100.

The obtaining unit 401 obtains, for example, a processing request requesting execution of quantum chemical calculation for a molecule of interest. The processing request may include, for example, structure information. The processing request may include, for example, a basis function system. The processing request may include, for example, the number of divisions. The processing request may include, for example, fragment information. Specifically, the obtaining unit 401 obtains the processing request by receiving an input of the processing request, based on an operation input of the user. For example, the obtaining unit 401 may obtain the processing request by receiving the processing request from another computer. The other computer is, for example, the client device 202.

The obtaining unit 401 obtains, for example, structure information. Specifically, the obtaining unit 401 obtains the structure information by receiving an input of the structure information, based on an operation input of the user. Specifically, the obtaining unit 401 may obtain the structure information by receiving the structure information from another computer. The other computer is, for example, the client device 202.

The obtaining unit 401 obtains, for example, a basis function system. Specifically, the obtaining unit 401 obtains the basis function system by receiving an input of the basis function system, based on an operation input of the user. Specifically, the obtaining unit 401 may obtain the basis function system by receiving the basis function system from another computer. The other computer is, for example, the client device 202.

The obtaining unit 401 obtains, for example, the number of divisions. Specifically, the obtaining unit 401 obtains the division number by receiving an input of the division number, based on an operation input of the user. Specifically, the obtaining unit 401 may obtain the division number by receiving the division number from another computer. The other computer is, for example, the client device 202.

The obtaining unit 401 obtains fragment information, for example. Specifically, the obtaining unit 401 obtains the fragment information by receiving an input of the fragment information based on an operation input of the user. Specifically, the obtaining unit 401 may obtain the fragment information by receiving the fragment information from another computer. The other computer is, for example, the client device 202.

The obtaining unit 401 may receive a start trigger for starting the process of any functional unit. The start trigger is, for example, a predetermined operation input by the user. The start trigger may be, for example, reception of predetermined information from another computer. The start trigger may be, for example, output of predetermined information by any functional unit. For example, the obtaining unit 401 regards obtaining a processing request as a start trigger for starting the processes of the dividing unit 402 and the iterating unit 403.

The dividing unit 402 divides the structure of the molecule of interest into multiple fragments corresponding to the number of divisions obtained by the obtaining unit 401 and based on the structure information obtained by the obtaining unit 401, thereby generating fragment information indicating each fragment. For example, the dividing unit 402 divides the structure of the molecule of interest into multiple fragments so that each fragment includes an atom other than hydrogen and a hydrogen atom directly connected to the atom other than hydrogen. Thus, the dividing unit 402 may reduce the workload on the user when generating fragment information.

The iterating unit 403 calculates the energy of the molecule of interest, based on the energy of each of the multiple fragments obtained by dividing the structure of the molecule of interest by the molecule dividing method. Examples of the molecule dividing method include BE and DMET. The iterating unit 403 sets a Hamiltonian corresponding to each of the multiple fragments based on, for example, the structure information, the basis function system, and the fragment information.

For example, the iterating unit 403 repeatedly performs a series of processes by the classifying unit 411, the first calculating unit 412, and the second calculating unit 413 until a predetermined exit condition is satisfied. The predetermined exit condition is set in advance by the user, for example. The predetermined exit condition is, for example, that the sum of the number of atoms of each fragment matches the number of atoms of the molecule. Thus, the iterating unit 403 may perform quantum chemical calculation for calculating the energy of the molecule of interest.

The classifying unit 411 classifies the fragments into multiple groups, based on the fragment information. For example, the classifying unit 411 calculates, for the multiple fragments, an index value related to the degree of similarity between Hamiltonians corresponding to a combination of two different fragments. For example, a smaller index value indicates a higher degree of similarity. For example, a larger index value may indicate a higher degree of similarity.

Specifically, the classifying unit 411 identifies, among the multiple fragments, a combination of two fragments corresponding to Hamiltonians in which all terms with creation/annihilation operators match each other. Specifically, the classifying unit 411 calculates an index value related to the degree of similarity between Hamiltonians corresponding to the identified combination based on an absolute difference value of coefficients related to the same term in the Hamiltonians corresponding to the identified combination. The index value is, for example, a statistical value of absolute difference values. The statistical value is, for example, a maximum value, a minimum value, an average value, a mode value, or a median value. Here, a smaller index value indicates a higher degree of similarity.

For example, the classifying unit 411 classifies multiple fragments into multiple groups, based on the calculated index value such that a combination of two different fragments corresponding to the same or similar Hamiltonians belongs to the same group. Specifically, the classifying unit 411 classifies multiple fragments into multiple groups such that, among the fragments, a combination in which the calculated index value is within a predetermined range belongs to the same group. For example, when a smaller index value indicates a higher degree of similarity, the predetermined range is specifically a range not more than a threshold value. The threshold is set in advance by the user, for example.

Accordingly, the classifying unit 411 may identify a group of fragments having similar properties with respect to the Hamiltonian. The classifying unit 411 may obtain a guideline for reducing the processing time necessary to calculate the energy of each fragment.

For a first fragment among the multiple fragments, the first calculating unit 412 calculates a first problem using a first Hamiltonian corresponding to the first fragment. The first problem is, for example, an eigenvalue problem. The first problem is, for example, a calculation process of obtaining an eigenstate corresponding to a wave function by solving the first Hamiltonian. The first problem is, for example, an intermediate calculation process for calculating the first energy corresponding to the first fragment. For the first fragment, the first calculating unit 412 calculates the first energy corresponding to the first fragment using the result of calculating the first problem.

For example, with respect to each of the groups classified by the classifying unit 411, the first calculating unit 412 selects any fragment belonging to the group as the first fragment. For example, for each group, the first calculating unit 412 calculates a first problem using a first Hamiltonian corresponding to the selected first fragment belonging to the group. For example, for each group, the first calculating unit 412 calculates the first energy corresponding to the selected first fragment belonging to the group by using the result of calculating the first problem.

Accordingly, the first calculating unit 412 may appropriately calculate, for each group, the first energy corresponding to at least one of the first fragments belonging to the group. In addition, the first calculating unit 412 may retain, for each group, a result of calculating the first problem that may be diverted when calculating the second energy corresponding to each second fragment belonging to the group and excluding the first fragment.

For a second fragment among the fragments, the second calculator 413 may calculate a second energy corresponding to the second fragment using a result of calculating the first problem. The second fragment is a fragment corresponding to a second Hamiltonian identical or similar to the first Hamiltonian among the fragments.

For example, with respect to each of the groups classified by the classifying unit 411, the second calculating unit 413 selects, as the second fragment, each fragment belonging to the group excluding the first fragment selected by the first calculating unit 412. For example, for each group, the second calculating unit 413 calculates the second energy corresponding to each selected second fragment belonging to the group by using the result of calculating the first problem by the first calculating unit 412.

Accordingly, the second calculating unit 413 may appropriately calculate, for each group, the second energy corresponding to each second fragment belonging to the group. The second calculating unit 413 may reduce the processing time and the processing load necessary to calculate the second energy corresponding to each second fragment by diverting the result of calculating the first problem.

The iterating unit 403 calculates the energy corresponding to the molecule of interest, based on the energies corresponding to the respective fragments calculated by the first calculating unit 412 and the second calculating unit 413. The iterating unit 403 calculates, for example, the total value of the calculated energies corresponding to the respective fragments as the energy corresponding to the molecule of interest. Accordingly, the iterating unit 403 may appropriately calculate the energy of the molecule of interest.

The iterating unit 403 determines whether the exit condition is satisfied when the first calculating unit 412 and the second calculating unit 413 calculate the energy corresponding to each fragment. The exit condition is, for example, that the sum of the number of atoms corresponding to each fragment matches the number of atoms corresponding to the molecule of interest. Accordingly, the iterating unit 403 may determine whether the energy corresponding to each fragment has been appropriately calculated.

The exit condition may be, for example, that a statistical value of a change amount between the energy of each fragment calculated this time and the energy of each fragment calculated the previous time is not more than a threshold value. The threshold is set in advance by the user, for example. The statistical value is, for example, a maximum value, a minimum value, an average value, a mode value, or a median value.

When the exit condition is not satisfied, the iterating unit 403 updates the Hamiltonian corresponding to each fragment. Accordingly, the iterating unit 403 may optimize the Hamiltonian corresponding to each fragment. The iterating unit 403 may allow the energy corresponding to each fragment to be recalculated.

The iterating unit 403 re-executes a series of processes by the classifying unit 411, the first calculating unit 412, and the second calculating unit 413 in response to updating the Hamiltonian corresponding to each fragment. Accordingly, the iterating unit 403 may optimize the energy corresponding to each fragment.

The output unit 404 outputs a processing result of at least one of the functional units. The output format is, for example, display on a display, print output to a printer, transmission to an external device by the network I/F 303, or storage in a storage area such as the memory 302 or the recording medium 305. Accordingly, the output unit 404 may notify the user of the processing result of at least one of the functional units, and the convenience of the information processing device 100 may be improved.

The output unit 404 outputs, for example, the energy corresponding to the molecule of interest calculated by the iterating unit 403. Specifically, the output unit 404 outputs the energy corresponding to the molecule of interest so that the user may refer to the energy. Specifically, the output unit 404 may transmit the energy corresponding to the molecule of interest to another computer. The other computer is, for example, the client device 202. Thus, the output unit 404 enables external reference of the energy corresponding to the molecule of interest.

The output unit 404 outputs, for example, the energy corresponding to each fragment calculated by the iterating unit 403. Specifically, the output unit 404 outputs the energy corresponding to each fragment so that the user may refer to the energy. Specifically, the output unit 404 may transmit the energy corresponding to each fragment to another computer. The other computer is, for example, the client device 202. Accordingly, the output unit 404 enables external reference of the energy corresponding to each fragment.

Here, while a case where the information processing device 100 includes the obtaining unit 401, the dividing unit 402, the iterating unit 403, and the output unit 404 has been described, the present disclosure is not limited hereto. For example, the information processing device 100 may omit any of the functional units. Specifically, the information processing device 100 may omit the dividing unit 402. In this case, specifically, the information processing device 100 may cooperate with another computer operating as the dividing unit 402. The other computer is, for example, the chemical calculation device 201.

Here, while a case where the first calculating unit 412 calculates the first energy corresponding to the first fragment after the classifying unit 411 classifies the multiple fragments into the multiple groups has been described, the present disclosure is not limited hereto. For example, after the first calculating unit 412 calculates the first energy corresponding to the first fragment, the classifying unit 411 may identify the second fragments whose Hamiltonians are the same as or similar to that of the first fragment.

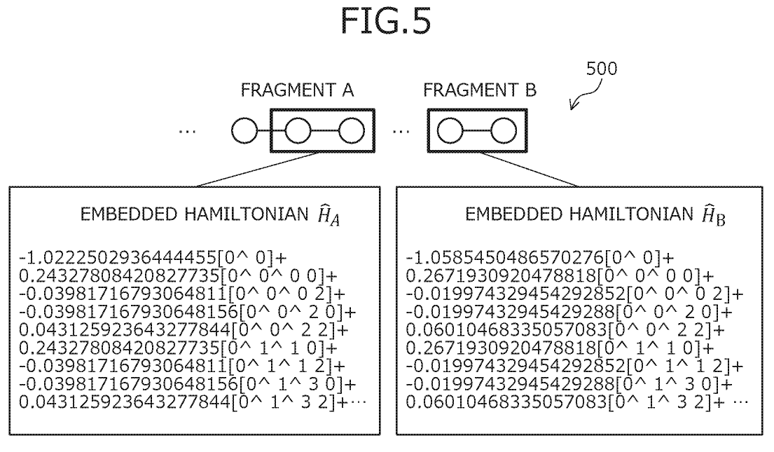

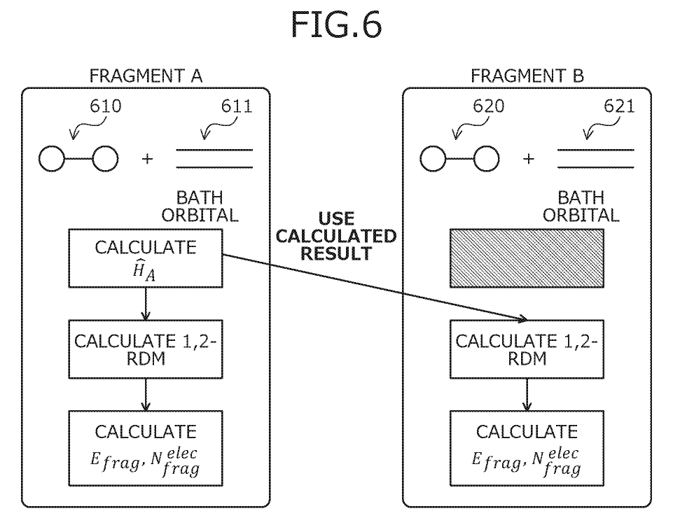

Next, an example of operation of the information processing device 100 will be described with reference to FIGS. 5 and 6. In FIGS. 5 and 6, the information processing device 100 calculates energy corresponding to a molecule of interest by DMET. The molecules of interest are, for example, clusters of hydrogen linked in chains.

FIGS. 5 and 6 are explanatory diagrams depicting an operation example of the information processing device 100. In FIG. 5, the information processing device 100 divides a structure 500 of a molecule of interest into multiple fragments. In the example depicted in FIG. 5, the multiple fragments specifically include a fragment A and a fragment B.

The information processing device 100 sets an embedded Hamiltonian corresponding to each fragment. In the example depicted in FIG. 5, specifically, the information processing device 100 sets an embedded Hamiltonian Ĥ_{A} corresponding to the fragment A. Specifically, the information processing device 100 sets an embedded Hamiltonian Ĥ_{B} corresponding to the fragment B. Here, for convenience, “H” with “ ̂” at the top thereof may be expressed as “H ̂”.

The embedded Hamiltonian is formed by multiple terms related to creation/annihilation operators and coefficients related to the terms, respectively. In the embedded Hamiltonian, [] represents a term related to a creation or annihilation operator. The value before [] of the embedded Hamiltonian represents the coefficient of the term. Here, with respect to embedded Hamiltonians having the same terms and similar coefficients applied to the respective terms, it is considered that results of calculating eigenvalue problems using the respective embedded Hamiltonians have similar properties.

The information processing device 100 uses the above properties to reduce the processing time necessary to perform quantum chemical calculation for calculating energy corresponding to a molecule of interest by DMET. For example, the information processing device 100 uses an intermediate calculation result when calculating energy corresponding to one fragment of two fragments corresponding to the same or similar embedded Hamiltonians when calculating energy corresponding to the other fragment. Here, FIG. 6 will be described.

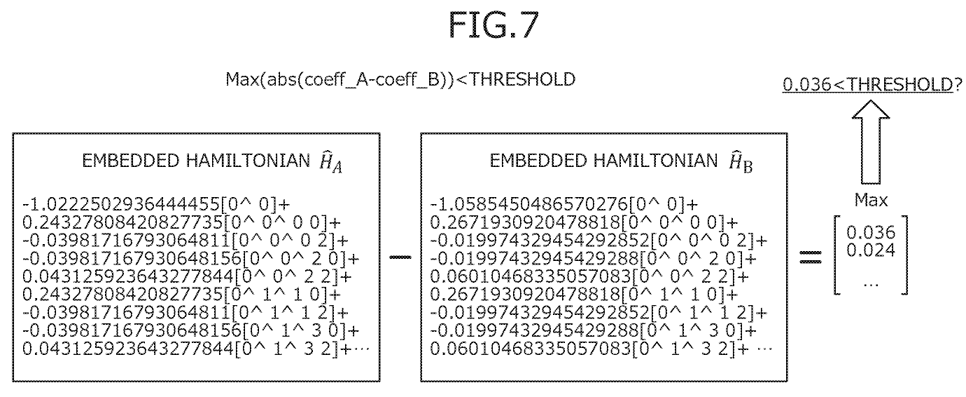

In FIG. 6, (6-1) the information processing device 100 classifies multiple fragments into multiple groups based on the embedded Hamiltonian corresponding to each fragment. For example, the information processing device 100 performs similarity determination of embedded Hamiltonians and identifies the same or similar embedded Hamiltonians. An example of the similarity determination of the embedded Hamiltonian will be described later with reference to FIG. 7.

For example, the information processing device 100 classifies multiple fragments into multiple groups so that a combination of two different fragments corresponding to the identified same or similar embedded Hamiltonians belongs to the same group. In the example depicted in FIG. 6, specifically, it is assumed that the information processing device 100 classifies multiple fragments into multiple groups so that a combination of a fragment A and a fragment B belongs to the same group. Here, FIG. 7 will be described.

FIG. 7 is an explanatory diagram depicting an example of similarity determination of embedded Hamiltonians. In FIG. 7, the information processing device 100 performs similarity determination between the embedded Hamiltonian Ĥ_{A} corresponding to the fragment A and the embedded Hamiltonian Ĥ_{B} corresponding to the fragment B.

(7-1) The information processing device 100 determines whether all terms of the creation/annihilation operator are common to the embedded Hamiltonian Ĥ_{A} and the embedded Hamiltonian Ĥ_{B}. The information processing device 100 determines that the embedded Hamiltonian Ĥ_{A} and the embedded Hamiltonian Ĥ_{B} are not similar to each other when at least one term of the creation/annihilation operators is not common. In the example depicted in FIG. 7, specifically, it is assumed that the information processing device 100 determines that all the terms of the creation/annihilation operator are common between the embedded Hamiltonian Ĥ_{A} and the embedded Hamiltonian Ĥ_{B}.

(7-2) When all the terms of the creation/annihilation operator are common, the information processing device 100 calculates the maximum value of the difference of absolute values of the coefficients related to the terms of the same creation/annihilation operator with respect to the embedded Hamiltonian Ĥ_{A} and the embedded Hamiltonian Ĥ_{B}. The maximum value is, for example, 0.036. When the calculated maximum value is at least equal to the threshold value, the information processing device 100 determines that the embedded Hamiltonian Ĥ_{A} and the embedded Hamiltonian Ĥ_{B} are not similar to each other.

On the other hand, when the calculated maximum value is less than the threshold value, the information processing device 100 determines that the embedded Hamiltonian Ĥ_{A} and the embedded Hamiltonian Ĥ_{B} are similar to each other. In the example depicted in FIG. 7, specifically, it is assumed that the information processing device 100 determines that the embedded Hamiltonian Ĥ_{A} and the embedded Hamiltonian Ĥ_{B} are similar to each other. Here, the description returns to FIG. 6.

(6-2) The information processing device 100 calculates, for each group, energy corresponding to any fragment belonging to the group. The information processing device 100 calculates, for each group, energy corresponding to each of other fragments belonging to the group by using an intermediate calculation result when energy corresponding to any fragment belonging to the group is calculated.

In the example depicted in FIG. 6, specifically, the information processing device 100 calculates an eigenvalue problem using the embedded Hamiltonian Ĥ_{A} for the fragment A based on a structure 610 of the fragment A and a bath orbital 611. Specifically, the information processing device 100 calculates 1,2-RDM, based on a result of calculating an eigenvalue problem using the embedded Hamiltonian Ĥ_{A}. Specifically, the information processing device 100 calculates the energy E_{frag} and the number of atoms N_{frag}^{elec} corresponding to the fragment A, based on the calculated 1,2-RDM.

Specifically, for the fragment B belonging to the same group as the fragment A, the information processing device 100 does not need to calculate the eigenvalue problem using the embedded Hamiltonian Ĥ_{B} based on the structure 620 of the fragment B and the bath orbital 621. Specifically, the information processing device 100 calculates 1,2-RDM by diverting a result of calculating an eigenvalue problem using the embedded Hamiltonian Ĥ_{A} for the fragment B. Specifically, the information processing device 100 calculates the energy E_{frag} and the number of atoms N_{frag}^{elec} corresponding to the fragment B based on the calculated 1,2-RDM.

(6-3) The information processing device 100 determines whether an exit condition is satisfied. The exit condition is that the total value of the number of atoms of each fragment matches the number of atoms of the molecule of interest. When the exit condition is not satisfied, the information processing device 100 updates a penalty value for the embedded Hamiltonian corresponding to each fragment and updates the embedded Hamiltonian corresponding to each fragment based on the penalty value.

When the embedded Hamiltonian corresponding to each fragment is updated, the information processing device 100 re-executes the series of processes (6-1) and (6-2). When the exit condition is satisfied, the information processing device 100 calculates and outputs the energy corresponding to the molecule of interest by adding the energies corresponding to the respective fragments.

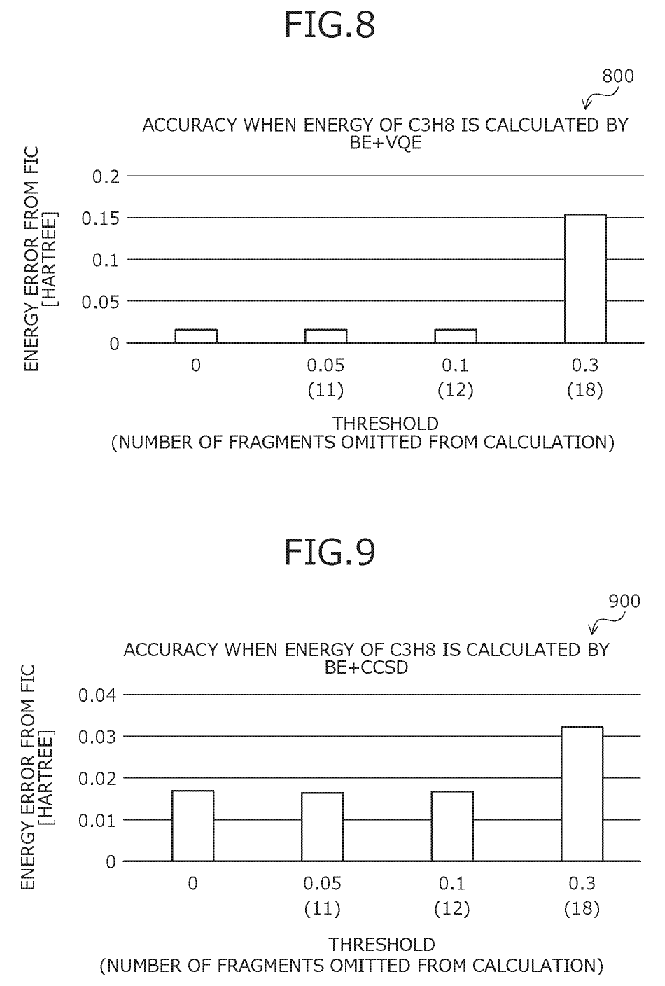

Accordingly, the information processing device 100 may appropriately calculate the energy corresponding to the molecule of interest. In addition, the information processing device 100 may reduce the processing time necessary to perform the quantum chemical calculation for calculating the energy corresponding to the molecule of interest. Next, an example of an effect of the information processing device 100 will be described with reference to FIGS. 8 and 9.

FIGS. 8 and 9 are explanatory diagrams depicting an example of an effect. In FIG. 8, it is assumed that the information processing device 100 calculates the energy corresponding to the molecule of interest C3H8 by BE to which VQE is applied in a case where the similarity determination threshold is 0, 0.05, 0.1, and 0.3.

Further, the horizontal axis of the graph 800 represents cases where the similarity determination threshold is 0, 0.05, 0.1, and 0.3, respectively. The parentheses on the horizontal axis of the graph 800 represent the number of fragments omitted without calculating the eigenvalue problem according to the similarity determination. The vertical axis of the graph 800 represents the accuracy of energy. Specifically, the vertical axis of the graph 800 represents an error from the energy corresponding to the molecule of interest C3H8 calculated by the FCI. Next, FIG. 9 will be described.

In FIG. 9, it is assumed that the information processing device 100 calculates the energy corresponding to the molecule of interest C3H8 by BE to which CCSD is applied when the similarity determination threshold is 0, 0.05, 0.1, and 0.3.

Further, the horizontal axis of the graph 900 represents cases where the similarity determination threshold is 0, 0.05, 0.1, and 0.3, respectively. The parentheses on the horizontal axis of the graph 900 represent the number of fragments omitted without calculating the eigenvalue problem according to the similarity determination. The vertical axis of the graph 900 represents the accuracy of energy. Specifically, the vertical axis of the graph 900 represents an error from the energy corresponding to the molecule of interest C3H8 calculated by the FCI.

As depicted in the graphs 800 and 900, the information processing device 100 may reduce the processing time necessary for performing the quantum chemical calculation while maintaining the accuracy of the energy corresponding to the molecule of interest C₃H₈ by appropriately setting the threshold for the similarity determination.

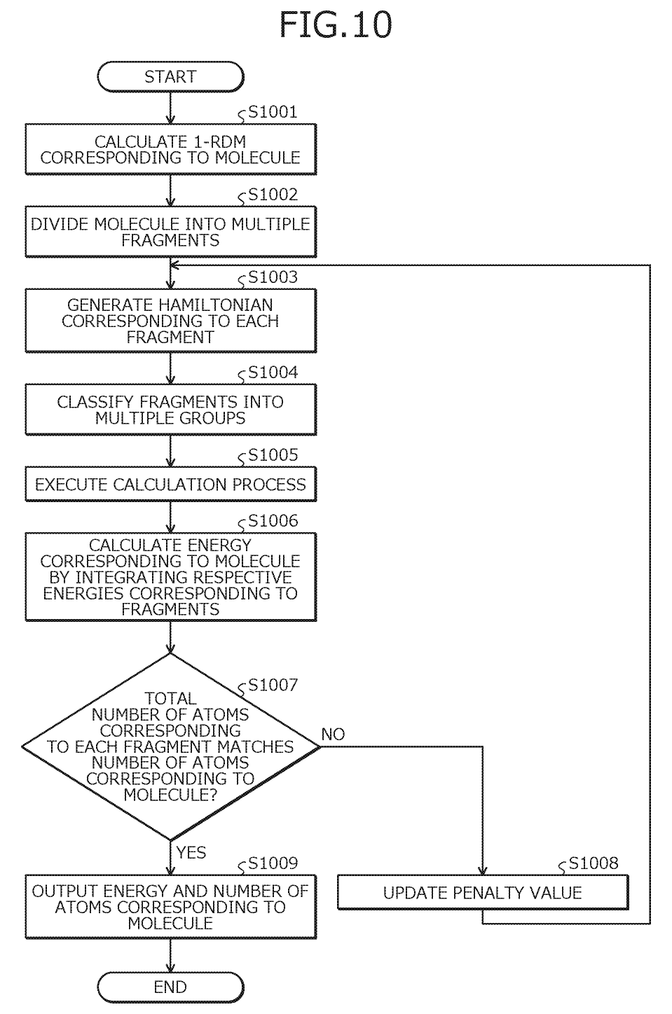

Next, an example of a procedure of an overall process executed by the information processing device 100 will be described with reference to FIG. 10. The overall process is implemented by, for example, the CPU 301, storage areas such as the memory 302 and the recording medium 305, and the network I/F 303 depicted in FIG. 3.

FIG. 10 is a flowchart depicting an example of the procedure of the overall process. In FIG. 10, the information processing device 100 calculates 1-RDM corresponding to a molecule (step S1001). Next, the information processing device 100 divides the molecule into multiple fragments (step S1002). Then, the information processing device 100 generates a Hamiltonian corresponding to each fragment (step S1003).

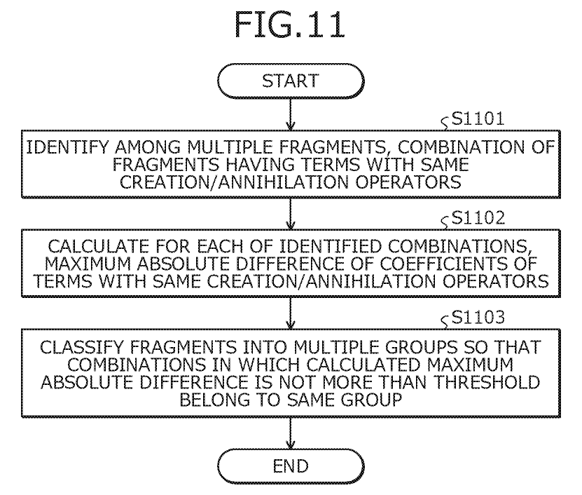

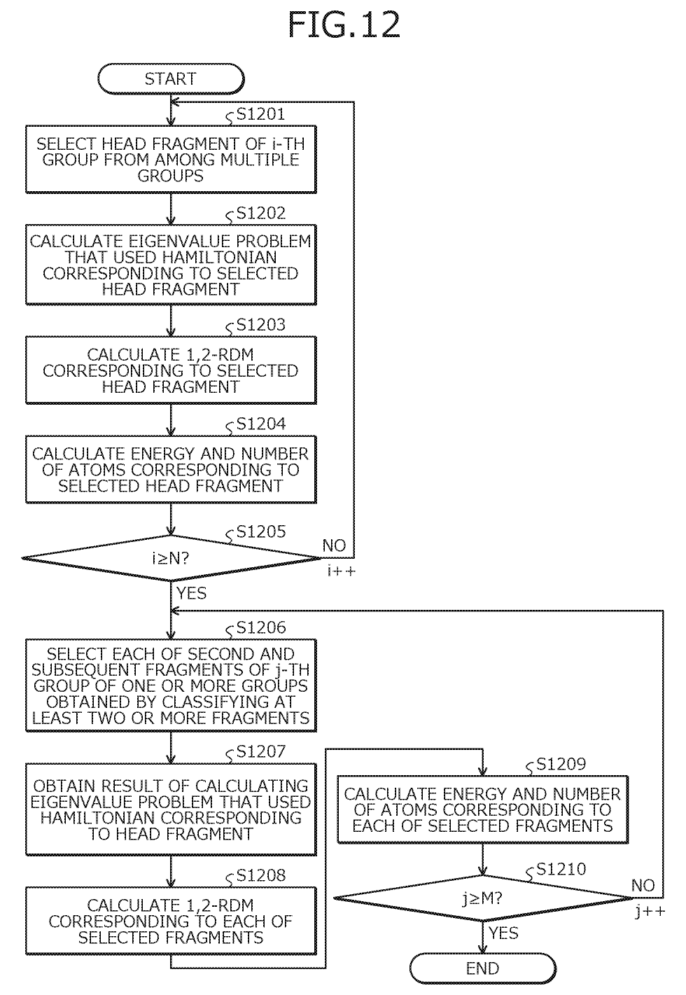

Next, the information processing device 100 classifies the fragments into multiple groups by executing a determination process described later with reference to FIG. 11 (step S1004). Then, the information processing device 100 calculates the energy corresponding to each fragment by executing a calculation process described later with reference to FIG. 12 (step S1005).

Next, the information processing device 100 calculates the energy corresponding to the molecule by integrating the respective energies corresponding to the fragments (step S1006). Then, the information processing device 100 determines whether the total number of atoms corresponding to each fragment matches the number of atoms corresponding to the molecule (step S1007).

When the numbers do not match (step S1007: NO), the information processing device 100 updates the penalty value (step S1008) and returns to the process at step S1003. On the other hand, when the numbers match (step S1007: YES), the information processing device 100 outputs the energy and the number of atoms corresponding to the molecule (step S1009) and ends the entire process. Accordingly, the information processing device 100 may perform quantum chemical calculation.

Next, an example of a procedure of the determination process executed by the information processing device 100 will be described with reference to FIG. 11. The determination process is implemented by, for example, the CPU 301, the storage area such as the memory 302 or the recording medium 305, and the network I/F 303 depicted in FIG. 3.

FIG. 11 is a flowchart depicting an example of the procedure of the determination process. In FIG. 11, the information processing device 100 identifies a combination of fragments having terms with the same creation/annihilation operators among multiple fragments (step S1101). Then, the information processing device 100 calculates for each of the identified combinations, the maximum absolute difference of the coefficients of the terms with the same creation/annihilation operators (step S1102).

Next, the information processing device 100 classifies the fragments into multiple groups so that combinations in which the calculated maximum absolute difference is not more than a threshold belong to the same group (step S1103). Then, the information processing device 100 ends the determination process. Accordingly, the information processing device 100 may group fragments having similar properties.

Next, an example of a procedure of the calculation process executed by the information processing device 100 will be described with reference to FIG. 12. The calculation process is implemented by, for example, the CPU 301, the storage area such as the memory 302 or the recording medium 305, and the network I/F 303 depicted in FIG. 3.

FIG. 12 is a flowchart depicting an example of the procedure of the calculation process. In FIG. 12, the information processing device 100 selects the head fragment of the i-th group from among multiple groups (step S1201). For example, the initial value of i is 1. For example, i is initialized each time calculation process is performed.

Next, the information processing device 100 calculates an eigenvalue problem that used a Hamiltonian corresponding to the selected head fragment (step S1202). Then, the information processing device 100 calculates 1,2-RDM corresponding to the selected head fragment on the basis of the result of calculating the eigenvalue problem using the Hamiltonian corresponding to the head fragment (step S1203).

Next, the information processing device 100 calculates the energy and the number of atoms corresponding to the selected head fragment, based on the 1,2-RDM (step S1204). Then, the information processing device 100 determines whether i≥N is satisfied (step S1205). For example, N is the total number of groups.

Here, when i<N true (step S1205: NO), the information processing device 100 increments i and returns to the process at step S1201. When i≥N is true (step S1205: YES), the information processing device 100 proceeds to the process at step S1206.

At step S1206, the information processing device 100 selects each of the second and subsequent fragments of a j-th group from one or more groups obtained by classifying at least two or more fragments among the multiple groups (step S1206). For example, the initial value of j is 1. For example, j is initialized each time calculation process is performed.

Next, the information processing device 100 obtains a result of calculating the eigenvalue problem that used the Hamiltonian corresponding to the head fragment of the j-th group (step S1207). Then, the information processing device 100 calculates 1,2-RDM corresponding to each of the selected fragments, based on the result of calculating the eigenvalue problem using the Hamiltonian corresponding to the head fragment of the j-th group (step S1208).

Next, the information processing device 100 calculates energy and the number of atoms corresponding to each of the selected fragments, based on the 1,2-RDM (step S1209). Then, the information processing device 100 determines whether j≥M is satisfied (step S1210). For example, M is the number of groups into which at least two or more fragments are classified.

Here, when j<M is true (step S1210: NO), the information processing device 100 returns to the process at step S1206. On the other hand, when j≥M is true (step S1210: YES), the information processing device 100 ends the calculation process. Accordingly, the information processing device 100 may calculate the energy corresponding to each fragment. The information processing device 100 may reduce the processing time necessary to calculate the energy corresponding to each fragment.

Here, the information processing device 100 may change the order of the processes of some steps of the flowcharts depicted in FIGS. 10 to 12. In addition, the information processing device 100 may omit the processes of some steps of the flowcharts depicted in FIGS. 10 to 12.

The information processing device 100 may be applied to fields such as drug discovery and material development. Specifically, in the field of drug discovery, material development, or the like, the information processing device 100 may be applied to a case where it is desired to perform quantum chemical calculation for calculating the basis energy of a molecule in order to analyze the structure or properties of the molecule, which is a candidate for a drug or a material. As a result, the information processing device 100 may reduce the processing time necessary to perform quantum chemical calculation, facilitate calculation of the basis energy of a molecule, and contribute to the fields of drug discovery, material development, and the like.

As described above, according to the information processing device 100, it is possible to calculate the energy of the molecule based on the energy of each fragment among multiple fragments obtained by dividing the structure of the molecule by the molecule dividing method. According to the information processing device 100, the first problem using the first Hamiltonian corresponding to the first fragment may be calculated for the first fragment among the multiple fragments. According to the information processing device 100, it is possible to calculate the first energy corresponding to the first fragment using the result of calculating the first problem. According to the information processing device 100, it is possible to identify a second fragment corresponding to a second Hamiltonian that is the same as or similar to the first Hamiltonian among the multiple fragments. According to the information processing device 100, for the second fragment, the second energy corresponding to the second fragment may be calculated using the result of calculating the first problem. The information processing device 100 may reduce the processing time necessary to perform the quantum chemical calculation.

The information processing device 100 may calculate, for the multiple fragments, an index value related to the degree of similarity between Hamiltonians corresponding to a combination of two different fragments. According to the information processing device 100, it is possible to classify fragments into multiple groups such that, among the multiple fragments, a combination in which the calculated index value is within a predetermined range belongs to the same group. According to the information processing device 100, for each group, any one of the first fragments belonging to the group may be selected. According to the information processing device 100, it is possible to calculate the first problem using the first Hamiltonian corresponding to the selected first fragment and calculate the first energy using the result of calculating the first problem. According to the information processing device 100, for each group, each second fragment belonging to the group, excluding the first fragment, may be selected. According to the information processing device 100, for the second fragment, the second energy may be calculated using the result of calculating the first problem. Accordingly, the information processing device 100 may classify multiple fragments into multiple groups such that two different fragments having similar properties belong to the same group. Therefore, the information processing device 100 may appropriately select the second fragment for reducing the processing time by diverting the result of calculating the first problem. As a result, the information processing device 100 may appropriately reduce the processing time necessary for performing the quantum chemical calculation.

According to the information processing device 100, it is possible to identify, among multiple fragments, a combination of two fragments corresponding to Hamiltonians in which all terms of creation/annihilation operators match each other. According to the information processing device 100, it is possible to calculate the index value related to the degree of similarity between the Hamiltonians corresponding to the identified combination, based on the difference absolute value of the coefficients related to the same term in the Hamiltonians corresponding to the identified combination. Accordingly, the information processing device 100 may accurately calculate the index value. Therefore, the information processing device 100 may easily classify multiple fragments into multiple groups so that two different fragments having similar properties belong to the same group.

According to the information processing device 100, the energy of the molecule may be calculated based on the calculated energy of each fragment. Thus, the information processing device 100 may complete the quantum chemical calculation for calculating the energy of the molecule.

According to the information processing device 100, in a case where the exit condition is not satisfied when the energy of each fragment is calculated, the Hamiltonian corresponding to each fragment may be updated. According to the information processing device 100, the process of classifying, the process of calculating the first energy, and the process of calculating the second energy may be re-executed in response to updating of the Hamiltonian corresponding to each fragment. Thus, the information processing device 100 may optimize the Hamiltonian and accurately calculate the energy of the molecule.

According to the information processing device 100, the structure of the molecule may be divided into multiple fragments. Thus, the information processing device 100 may identify multiple fragments by itself. The information processing device 100 may reduce the workload on the user when dividing the molecular structure into multiple fragments.

According to the information processing device 100, it is possible to calculate the first problem that is a calculation process of obtaining an eigenstate corresponding to a wave function. Accordingly, the information processing device 100 may calculate the energy of the fragment.

According to the information processing device 100, DMET may be employed as the molecule dividing method. Accordingly, the information processing device 100 may reduce the processing time necessary to perform the quantum chemical calculation for calculating the energy of the molecule by DMET.

The information processing method described in the present embodiment may be implemented by executing a prepared program on a computer such as a personal computer and a workstation. The program is stored on a non-transitory, computer-readable recording medium such as a hard disk, a flexible disk, a compact disc read-only memory (CD-ROM), a magneto-optical (MO) disc, and a digital versatile disc (DVD), read out from the computer-readable medium, and executed by the computer. The program may be distributed through a network such as the Internet.

According to the information processing device 100, it is possible to reduce the processing time necessary when quantum chemical calculation is performed using the molecule dividing method.

All examples and conditional language provided herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A computer-readable recording medium storing therein a program for causing a computer to execute a process, the process comprising:
calculating an energy of a molecule, based on an energy of each of a plurality of fragments obtained by dividing a structure of the molecule by a molecule dividing method, the calculating the energy including:
calculating, for a first fragment among the plurality of fragments, a first problem using a first Hamiltonian corresponding to the first fragment, and calculating a first energy corresponding to the first fragment using a result of calculating the first problem; and
calculating, for a second fragment among the plurality of fragments and corresponding to a second Hamiltonian identical or similar to the first Hamiltonian, a second energy corresponding to the second fragment using a result of calculating the first problem.

2. The computer-readable recording medium according to claim 1, the process further comprising:
calculating, for the plurality of fragments, an index value related to a degree of similarity between Hamiltonians respectively corresponding to two different fragments of a combination of the plurality of fragments; and
classifying the plurality of fragments into a plurality of groups so that among the plurality of fragments, combinations thereof for which the calculated index value is within a predetermined range belong to a same group, wherein
the calculating the first energy includes calculating, with respect to each of the plurality of groups, a first problem using a first Hamiltonian corresponding to any one of first fragments belonging to the each of the plurality of groups, and using a result of calculating the first problem to calculate the first energy, and
the calculating the second energy includes calculating, with respect to each of the plurality of groups, the second energy using a result of calculating the first problem, the second energy being calculated for each second fragment belonging to the each of the plurality of groups and exclusive of the first fragment.

3. The computer-readable recording medium according to claim 2, wherein
the classifying includes identifying among the plurality of fragments, a combination of two fragments corresponding to Hamiltonians in which all terms with a generation/annihilation operator match each other, and
the calculating the index value includes calculating the index value related to the degree of similarity between the Hamiltonians corresponding to the identified combination, based on an absolute difference value of coefficients related to a same term in each of the Hamiltonians corresponding to the identified combination.

4. The recording medium according to claim 3, wherein the calculating the energy of the molecule includes calculating the energy based on the calculated energy of each of the plurality of fragments.

5. The computer-readable recording medium according to claim 2, the process further comprising updating the Hamiltonians respectively corresponding to the plurality of fragments when an exit condition is not satisfied when the energy of each of the plurality of fragments is calculated, wherein
the classifying, the calculating the first energy, and the calculating the second energy are re-executed in response to updating the Hamiltonians respectively corresponding to the plurality of fragments.

6. The recording medium according to claim 1, the process further comprising dividing a structure of the molecule into the plurality of fragments.

7. The computer-readable recording medium according to claim 1, wherein the first problem is a calculation process of obtaining an eigenstate corresponding to a wave function.

8. The computer-readable recording medium according to claim 1, wherein the molecule dividing method is Density Matrix Embedding Theory or Bootstrap Embedding.

9. An information processing method executed by a computer, the method comprising:
calculating an energy of a molecule, based on an energy of each of a plurality of fragments obtained by dividing a structure of the molecule by a molecule dividing method, the calculating the energy including:
calculating, for a first fragment among the plurality of fragments, a first problem using a first Hamiltonian corresponding to the first fragment, and calculating a first energy corresponding to the first fragment using a result of calculating the first problem; and
calculating, for a second fragment among the plurality of fragments and corresponding to a second Hamiltonian identical or similar to the first Hamiltonian, a second energy corresponding to the second fragment using a result of calculating the first problem.

10. An information processing device, comprising:
a memory; and
a processor coupled to the memory, the processor configured to:
calculate an energy of a molecule, based on an energy of each of a plurality of fragments obtained by dividing a structure of the molecule by a molecule dividing method, the calculating the energy including:
calculate, for a first fragment among the plurality of fragments, a first problem using a first Hamiltonian corresponding to the first fragment, and calculating a first energy corresponding to the first fragment using a result of calculating the first problem; and
calculate, for a second fragment among the plurality of fragments and corresponding to a second Hamiltonian identical or similar to the first Hamiltonian, a second energy corresponding to the second fragment using a result of calculating the first problem.
